(19) United States
(12) Patent Application Publication
(10) Pub. No.: US 2021/0403441 A1
(43) Pub. Date: Dec. 30, 2021

(54) FUNCTIONAL GROUP-PROTECTED DIAZIDOGLYOXIME, METHOD OF SYNTHESIZING THE SAME, AND METHOD OF SYNTHESIZING TKX-50 USING FUNCTIONAL GROUP-PROTECTED DIAZIDOGLYOXIME

(71) Applicant: Agency for Defense Development, Daejon (KR)

(72) Inventors: Kuk Tae Kwon, Daejeon (KR); Seung Hee Kim, Daejeon (KR); Woong Hee Lee, Daejeon (KR); Jeong Sub Shim, Daejeon (KR)

(21) Appl. No.: 16/975,084

(22) PCT Filed: Jan. 10, 2020
(86) PCT No.: PCT/KR2020/000445

§ 371 Date: Aug. 21, 2020

(30) Foreign Application Priority Data
Oct. 4, 2019 (KR) 10-2019-0123162
Oct. 17, 2019 (KR) 10-2019-0129181

(51) Int. Cl. C07D 257/04; C06B 25/34

(52) U.S. Cl. CPC C07D 257/04; C06B 25/34

(57) ABSTRACT

The present invention relates to functional group-protected diazidoglyoxime (DAG), a method of synthesizing the same, and a method of synthesizing TKX-50 using functional group-protected DAG. Insensitive-DAG with enhanced insensitivity, instead of sensitive DAG, may be synthesized to reduce risks of processes and harmfulness arising from threats of explosion and fire accidents caused by impact, friction and static electricity, and thus it is possible to stably synthesize functional group-protected DAG.

### TECHNICAL FIELD

The present invention relates to functional group-protected diazidoglyoxime, a method of synthesizing the same, and a method of synthesizing TKX-50 USING functional group-protected diazidoglyoxime.

### BACKGROUND ART

Currently, the most widely used high-energy materials for military explosive are 1,3,5-trinitro-1,3,5-triazacyclohexane (RDX), 1,3,5,7-tetranitro-1,3,5,7-tetraazacyclooctane (HMX), 2,4,6,8,10,12-hexanitro-2,4,6,8,10,12-hexaza-isowurtzitane (CL-20), and the like, and are being used in a wide variety of fields. Recently, with the development of new weapon systems, many studies are being conducted to develop high-energy materials with higher explosive performance than high-energy materials that are previously used. In particular, research on materials containing ring or cage structures has been actively conducted. Among the developed high-energy materials, dinitroazofuroxane (DDF) and octanitrocubane (ONC) have a very excellent explosive performance with a detonation velocity of about 10,000 m/s. However, since the DDF and ONC are very sensitive, there is a critical drawback of threatening a safety of a person handling the DDF and ONC.

Recently, to enhance an explosive performance and insensitivity, research is being actively conducted on cyclic compounds with a high nitrogen content such as triazole, tetrazole, nitroiminotetrazole, tetrazine, and the like. Among such cyclic compounds, dihydroxylammonium 5,5′-bistetrazole-1,1′-diolate (TKX-50) that is a tetrazole compound is regarded as one of promising high-energy materials. TKX-50 is known to have a higher explosive performance than those of existing energy materials (RDX, HMX, CL-20), and also to be insensitive.

FIG. 1 illustrates an existing method of synthesizing TKX-50. Referring to FIG. 1, the existing method of synthesizing TKX-50 is performed through five synthesis steps using glyoxal as a starting material. TKX-50 itself is insensitive in comparison to existing high-energy materials, but diazidoglyoxime (DAG), which is an intermediate obtained after an azidation reaction that introduces an energy group, has a very high sensitivity (DAG: impact sensitivity of 1.5 J, friction sensitivity of 5 N or less, and electrostatic sensitivity of 7 mJ) at a level of a primary explosive (lead styphnate: impact sensitivity of 2.5 to 5 J and friction sensitivity of 1.5 N, and lead azide: impact sensitivity of 2.5 to 4 J and friction sensitivity of 0.1 to 1 N). Accordingly, the DAG threatens a safety of an operator during a synthesis of TKX-50 and also has a danger of an accident.

Since hydrogen chloride (HCl) gas is used in a last step of the known synthesis of TKX-50, use of the HCl gas poses a safety risk and it is difficult to apply an effective process.

### DETAILED DISCLOSURE OF THE INVENTION

### Technical Subject

One or more example embodiments of the present invention are to solve the aforementioned problems, and an aspect of the present invention is to provide functional group-protected diazidoglyoxime (DAG) and a method of synthesizing the same that may synthesize relatively insensitive R-DAG, instead of sensitive DAG, safely from threats of explosion and fire accidents caused by impact, friction and static electricity, and that may utilize a synthesized material to synthesize various materials.

Another aspect of the present invention is to provide a method of synthesizing TKX-50 using functional group-protected DAG that may synthesize TKX-50 through relatively insensitive O,O′-ditetrahydropyranyloxalohydroximoyl diazide (THP-DAG), instead of DAG that is a sensitive intermediate so that an operator may more safely and effectively work during a synthesis of TKX-50, and that may use an aqueous HCl solution instead of HCl gas.

However, the problems to be solved by the present invention are not limited to the aforementioned problems, and other problems to be solved, which are not mentioned above, will be clearly understood by a person having ordinary skill in the art from the following description.

### Technical Solution

According to an example embodiment of the present invention, there is provided functional group-protected diazidoglyoxime (DAG) represented by the following Chemical Formula 1:

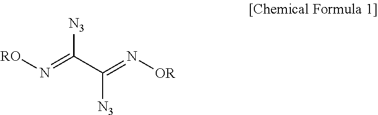

(Here, R includes at least one selected from the group consisting of tetrahydropyranyl (THP), methyl (Me), methoxymethyl (MOM), methoxythiomethyl (MTM), benzyloxymethyl (BOM), 2-methoxymethyl (MEM), 2-(trimethylsilyl)ethoxymethyl (SEM), tetrahydrofuranyl (THF), t-butyl, allyl, benzyl, p-methoxybenzyl, 3,4-dimethoxybenzyl, o-nitrobenzyl, p-nitrobenzyl, triphenylmethyl, trimethylsilyl (TMS), triethylsilyl (TES), triisopropylsilyl (TIPS), t-butyldimethylsilyl (TBDMS), t-butyldiphenylsilyl (TBDPS), diphenylmethylsilyl (DPMS), di-t-butylmethylsilyl (DTBMS), acetate, chloroacetate, methoxyacetate, triphenylmethoxyacetate, pivaloate, benzoate, and p-toluenesulfonate (Ts)).

In one example embodiment, the functional group-protected DAG may have an impact sensitivity of 1.5 J to 19 J, a friction sensitivity of 5 N to 350 N, and an electrostatic sensitivity of 7 mJ to 50 mJ.

In one example embodiment, the functional group-protected DAG may be synthesized from dichloroglyoxime (DCG).

In one example embodiment, the functional group-protected DAG may be synthesized from R-DCG that is synthesized from DCG and that is represented by the following Chemical Formula 2:

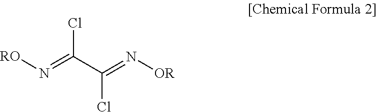

(Here, R includes at least one selected from the group consisting of tetrahydropyranyl (THP), methyl (Me), methoxymethyl (MOM), methoxythiomethyl (MTM), benzyloxymethyl (BOM), 2-methoxymethyl (MEM), 2-(trimethylsilyl)ethoxymethyl (SEM), tetrahydrofuranyl (THF), t-butyl, allyl, benzyl, p-methoxybenzyl, 3,4-dimethoxybenzyl, o-nitrobenzyl, p-nitrobenzyl, triphenylmethyl, trimethylsilyl (TMS), triethylsilyl (TES), triisopropylsilyl (TIPS), t-butyldimethylsilyl (TBDMS), t-butyldiphenylsilyl (TBDPS), diphenylmethylsilyl (DPMS), di-t-butylmethylsilyl (DTBMS), acetate, chloroacetate, methoxyacetate, triphenylmethoxyacetate, pivaloate, benzoate, and p-toluenesulfonate (Ts)).

In one example embodiment, the functional group-protected DAG may be an intermediate for preparation of one selected from the group consisting of an insensitive explosive, a non-toxic low-temperature gas generator, low-lead and/or lead-free pyrotechnics, and pharmaceutical chemicals.

In one example embodiment, the insensitive explosive may be dihydroxylammonium 5,5′-bistetrazole-1,1′-diolate (TKX-50).

According to another example embodiment of the present invention, there is provided a method of synthesizing functional group-protected DAG, the method including preparing DCG as a starting material; and forming R-DAG from the DCG, the R-DAG being represented by the following Chemical Formula 1:

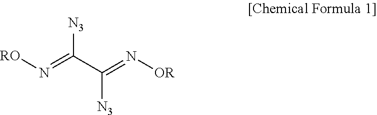

(Here, R includes at least one selected from the group consisting of tetrahydropyranyl (THP), methyl (Me), methoxymethyl (MOM), methoxythiomethyl (MTM), benzyloxymethyl (BOM), 2-methoxymethyl (MEM), 2-(trimethylsilyl)ethoxymethyl (SEM), tetrahydrofuranyl (THF), t-butyl, allyl, benzyl, p-methoxybenzyl, 3,4-dimethoxybenzyl, o-nitrobenzyl, p-nitrobenzyl, triphenylmethyl, trimethylsilyl (TMS), triethylsilyl (TES), triisopropylsilyl (TIPS), t-butyldimethylsilyl (TBDMS), t-butyldiphenylsilyl (TBDPS), diphenylmethylsilyl (DPMS), di-t-butylmethylsilyl (DTBMS), acetate, chloroacetate, methoxyacetate, triphenylmethoxyacetate, pivaloate, benzoate, and p-toluenesulfonate (Ts)).

In one example embodiment, the R-DAG may have an impact sensitivity of 1.5 J to 19 J, a friction sensitivity of 5 N to 350 N, and an electrostatic sensitivity of 7 mJ to 50 mJ.

In one example embodiment, the method may include synthesizing dichloroglyoxime (DCG); synthesizing R-DCG through the DCG, the R-DCG being represented by the following Chemical Formula 2; and synthesizing R-DAG through the R-DCG:

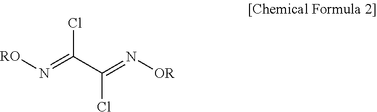

(Here, R includes at least one selected from the group consisting of tetrahydropyranyl (THP), methyl (Me), methoxymethyl (MOM), methoxythiomethyl (MTM), benzyloxymethyl (BOM), 2-methoxymethyl (MEM), 2-(trimethylsilyl)ethoxymethyl (SEM), tetrahydrofuranyl (THF), t-butyl, allyl, benzyl, p-methoxybenzyl, 3,4-dimethoxybenzyl, o-nitrobenzyl, p-nitrobenzyl, triphenylmethyl, trimethylsilyl (TMS), triethylsilyl (TES), triisopropylsilyl (TIPS), t-butyldimethylsilyl (TBDMS), t-butyldiphenylsilyl (TBDPS), diphenylmethylsilyl (DPMS), di-t-butylmethylsilyl (DTBMS), acetate, chloroacetate, methoxyacetate, triphenylmethoxyacetate, pivaloate, benzoate, and p-toluenesulfonate (Ts)).

In one example embodiment, the synthesizing of the dichloroglyoxime (DCG) may include synthesizing glyoxime; and reacting the glyoxime with N-chlorosuccinimide.

In one example embodiment, the synthesizing of the R-DCG through the DCG may be performed by reacting the DCG with a compound including at least one selected from the group consisting of tetrahydropyranyl (THP), methyl (Me), methoxymethyl (MOM), methoxythiomethyl (MTM), benzyloxymethyl (BOM), 2-methoxymethyl (MEM), 2-(trimethylsilyl)ethoxymethyl (SEM), tetrahydrofuranyl (THF), t-butyl, allyl, benzyl, p-methoxybenzyl, 3,4-dimethoxybenzyl, o-nitrobenzyl, p-nitrobenzyl, triphenylmethyl, trimethylsilyl (TMS), triethylsilyl (TES), triisopropylsilyl (TIPS), t-butyldimethylsilyl (TBDMS), t-butyldiphenylsilyl (TBDPS), diphenylmethylsilyl (DPMS), di-t-butylmethylsilyl (DTBMS), acetate, chloroacetate, methoxyacetate, triphenylmethoxyacetate, pivaloate, benzoate, and p-toluenesulfonate (Ts).

In one example embodiment, the synthesizing of the R-DCG through the DCG may be performed in the presence of a pyridinium p-toluenesulfonate (PPTS) catalyst.

In one example embodiment, the synthesizing of the R-DCG through the DCG may be performed by stirring and reacting the DCG, the PPTS catalyst, and the compound at a molar ratio of 0.5 to 2:0.02 to 0.5:3 to 7.

In one example embodiment, the stirring may be performed at a temperature of room temperature to 60° C.

In one example embodiment, the synthesizing of the R-DAG through the R-DCG may be performed through an azidation reaction.

In one example embodiment, the synthesizing of the R-DAG through the R-DCG may be performed by reacting the R-DCG with sodium azide (NaN₃).

In one example embodiment, the synthesizing of the R-DAG through the R-DCG may be performed by stirring and reacting the R-DCG and the sodium azide at a molar ratio of 1:2 to 4.

In one example embodiment, the stirring may be performed at a temperature of 95° C. to 100° C.

According to another example embodiment of the present invention, there is provided a method of synthesizing TKX-50 using functional group-protected DAG, the method including preparing DCG as a starting material; forming an insensitive-DAG intermediate from the DCG, the insensitive-DAG intermediate being represented by the following Chemical Formula 1; and synthesizing TKX-50 through the insensitive-DAG intermediate:

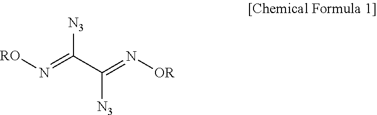

(Here, R includes at least one selected from the group consisting of tetrahydropyranyl (THP), methyl (Me), methoxymethyl (MOM), methoxythiomethyl (MTM), benzyloxymethyl (BOM), 2-methoxymethyl (MEM), 2-(trimethylsilyl)ethoxymethyl (SEM), tetrahydrofuranyl (THF), t-butyl, allyl, benzyl, p-methoxybenzyl, 3,4-dimethoxybenzyl, o-nitrobenzyl, p-nitrobenzyl, triphenylmethyl, trimethylsilyl (TMS), triethylsilyl (TES), triisopropylsilyl (TIPS), t-butyldimethylsilyl (TBDMS), t-butyldiphenylsilyl (TBDPS), diphenylmethylsilyl (DPMS), di-t-butylmethylsilyl (DTBMS), acetate, chloroacetate, methoxyacetate, triphenylmethoxyacetate, pivaloate, benzoate, and p-toluenesulfonate (Ts)).

In one example embodiment, the TKX-50 may be free of diazidoglyoxime (DAG) that is an intermediate byproduct.

In one example embodiment, the insensitive-DAG intermediate may have an impact sensitivity of 1.5 J to 19 J, a friction sensitivity of 5 N to 350 N, and an electrostatic sensitivity of 7 mJ to 50 mJ.

In one example embodiment, the method may include synthesizing dichloroglyoxime (DCG); synthesizing an R-DCG intermediate through the DCG, the R-DCG intermediate being represented by the following Chemical Formula 2; synthesizing an insensitive-DAG intermediate through the R-DCG intermediate; and synthesizing TKX-50 through the insensitive-DAG intermediate:

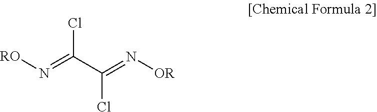

(Here, R includes at least one selected from the group consisting of tetrahydropyranyl (THP), methyl (Me), methoxymethyl (MOM), methoxythiomethyl (MTM), benzyloxymethyl (BOM), 2-methoxymethyl (MEM), 2-(trimethylsilyl)ethoxymethyl (SEM), tetrahydrofuranyl (THF), t-butyl, allyl, benzyl, p-methoxybenzyl, 3,4-dimethoxybenzyl, o-nitrobenzyl, p-nitrobenzyl, triphenylmethyl, trimethylsilyl (TMS), triethylsilyl (TES), triisopropylsilyl (TIPS), t-butyldimethylsilyl (TBDMS), t-butyldiphenylsilyl (TBDPS), diphenylmethylsilyl (DPMS), di-t-butylmethylsilyl (DTBMS), acetate, chloroacetate, methoxyacetate, triphenylmethoxyacetate, pivaloate, benzoate, and p-toluenesulfonate (Ts)). In one example embodiment, the synthesizing of the dichloroglyoxime (DCG) may include synthesizing glyoxime; and reacting the glyoxime with N-chlorosuccinimide.

In one example embodiment, the synthesizing of the R-DCG intermediate through the DCG may be performed by reacting the DCG with a compound including at least one selected from the group consisting of tetrahydropyranyl (THP), methyl (Me), methoxymethyl (MOM), methoxythiomethyl (MTM), benzyloxymethyl (BOM), 2-methoxymethyl (MEM), 2-(trimethylsilyl)ethoxymethyl (SEM), tetrahydrofuranyl (THF), t-butyl, allyl, benzyl, p-methoxybenzyl, 3,4-dimethoxybenzyl, o-nitrobenzyl, p-nitrobenzyl, triphenylmethyl, trimethylsilyl (TMS), triethylsilyl (TES), triisopropylsilyl (TIPS), t-butyldimethylsilyl (TBDMS), t-butyldiphenylsilyl (TBDPS), diphenylmethylsilyl (DPMS), di-t-butylmethylsilyl (DTBMS), acetate, chloroacetate, methoxyacetate, triphenylmethoxyacetate, pivaloate, benzoate, and p-toluenesulfonate (Ts).

In one example embodiment, the synthesizing of the R-DCG intermediate through the DCG may be performed in the presence of a pyridinium p-toluenesulfonate (PPTS) catalyst.

In one example embodiment, the synthesizing of the R-DCG through the DCG may be performed by stirring and reacting the DCG, the PPTS catalyst, and the compound at a molar ratio of 1:0.1:5.

In one example embodiment, the stirring of the DCG, the PPTS catalyst, and the compound may be performed at a temperature of room temperature to 60° C.

In one example embodiment, the synthesizing of the insensitive-DAG intermediate through the R-DCG intermediate may be performed through an azidation reaction.

In one example embodiment, the synthesizing of the insensitive-DAG intermediate through the R-DCG intermediate may be performed by reacting the R-DCG intermediate with sodium azide (NaN₃).

In one example embodiment, the synthesizing of the insensitive-DAG intermediate through the R-DCG intermediate may be performed by stirring and reacting the R-DCG intermediate and the sodium azide may be stirred at a molar ratio of 1:2 to 4.

In one example embodiment, the stirring of the R-DCG intermediate and the sodium azide may be performed at a temperature of 95° C. to 100° C. In one example embodiment, the synthesizing of the TKX-50 through the insensitive-DAG intermediate may include synthesizing 5,5′-bistetrazole-1,1′-diol by reacting the insensitive-DAG intermediate with an aqueous hydrochloric acid solution; and synthesizing the TKX-50 by reacting the 5,5′-bistetrazole-1,1′-diol with hydroxylamine.

In one example embodiment, the synthesizing of the 5,5′-bistetrazole-1,1′-diol by reacting the insensitive-DAG intermediate with the aqueous hydrochloric acid solution may be performed by stirring the insensitive-DAG intermediate and the aqueous hydrochloric acid solution under a temperature condition of room temperature.

In one example embodiment, the synthesizing of the TKX-50 by reacting the 5,5′-bistetrazole-1,1′-diol with the hydroxylamine may be performed by stirring and reacting the 5,5′-bistetrazole-1,1′-diol and the hydroxylamine at a molar ratio of 1:3 to 50. In one example embodiment, the stirring of the 5,5′-bistetrazole-1,1′-diol and the hydroxylamine may be performed at a temperature of 40° C. to 60° C.

### Effect of the Invention

According to example embodiments of the present invention, by functional group-protected diazidoglyoxime (DAG) and a method of synthesizing the same, R-DAG with enhanced insensitivity, instead of sensitive DAG, may be synthesized to reduce risks of processes and harmfulness arising from threats of explosion and fire accidents caused by impact, friction and static electricity, and thus it is possible to stably synthesize functional group-protected DAG. Also, it is possible to use the synthesized functional group-protected DAG as an intermediate to synthesize various materials. According to example embodiments of the present invention, by a method of synthesizing TKX-50 using functional group-protected DAG, it is possible to work safely from threats of explosion and fire accidents caused by impact, friction and static electricity using an insensitive-DAG intermediate that is an intermediate with enhanced insensitivity instead of DAG that is a sensitive intermediate synthesized during a synthesis of TKX-50, in comparison to a synthesis method according to a related art. Also, by using an aqueous HCl solution instead of HCl gas, it is possible to easily and safely perform a process.

### BRIEF DESCRIPTION OF THE DRAWINGS

FIG. 1 illustrates an existing method of synthesizing TKX-50.

FIG. 2 is a diagram illustrating a method of synthesizing functional group-protected diazidoglyoxime (DAG) according to an example embodiment of the present invention.

FIG. 3 is a diagram illustrating a method of synthesizing TKX-50 using functional group-protected DAG according to an example embodiment of the present invention.

FIG. 4 is a diagram illustrating a synthesis method of THP-DAG synthesized in Examples 1 to 4 according to the present invention.

FIG. 5 illustrates nuclear magnetic resonance (NMR) graphs of glyoxime synthesized in Example 1 of the present invention.

FIG. 6 illustrates NMR graphs of DCG synthesized in Example 2 of the present invention.

FIG. 7 illustrates NMR graphs of THP-DCG synthesized in Example 3 of the present invention.

FIG. 8 illustrates NMR graphs of THP-DAG synthesized in Example 4 of the present invention.

FIG. 9 illustrates NMR graphs of TKX-50 synthesized in Example 5 of the present invention.

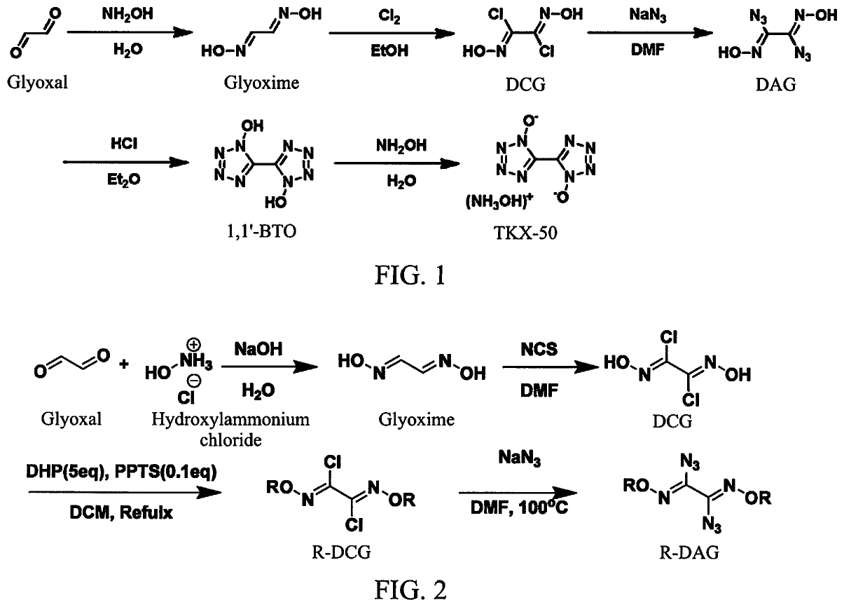

### BEST MODE FOR CARRYING OUT THE INVENTION

Hereinafter, example embodiments of the present invention will be described in detail with reference to the accompanying drawings. When it is determined detailed description related to a related known function or configuration they may make the purpose of the present invention unnecessarily ambiguous in describing the present invention, the detailed description will be omitted here. Also, terminologies used herein are defined to appropriately describe the example embodiments and thus may be changed depending on a user, the intent of an operator, or a custom of a field to which the present invention pertains. Accordingly, the terminologies must be defined based on the following overall description of the present specification. The same reference numerals as shown in each drawing represent same elements.

Throughout the specification, when any element is positioned “on” the other element, this not only includes a case that the any element is brought into contact with the other element, but also includes a case that another element exists between two elements.

Throughout the specification, if a prescribed part “includes” a prescribed element, this means that another element can be further included instead of excluding other elements unless any particularly opposite description exists.

Hereinafter, functional group-protected diazidoglyoxime (DAG) (that may be hereinafter referred to as “R-DAG”), a method of synthesizing the same, and a method of synthesizing TKX-50 using the functional group-protected DAG will be described in detail with reference to example embodiments and drawings. However, the present invention is not limited to the example embodiments and drawings.

According to an example embodiment of the present invention, the functional group-protected DAG may be represented by the following Chemical Formula 1:

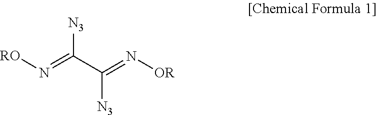

(Here, R includes at least one selected from the group consisting of tetrahydropyranyl (THP), methyl (Me), methoxymethyl (MOM), methoxythiomethyl (MTM), benzyloxymethyl (BOM), 2-methoxymethyl (MEM), 2-(trimethylsilyl)ethoxymethyl (SEM), tetrahydrofuranyl (THF), t-butyl, allyl, benzyl, p-methoxybenzyl, 3,4-dimethoxybenzyl, o-nitrobenzyl, p-nitrobenzyl, triphenylmethyl, trimethylsilyl (TMS), triethylsilyl (TES), triisopropylsilyl (TIPS), t-butyldimethylsilyl (TBDMS), t-butyldiphenylsilyl (TBDPS), diphenylmethylsilyl (DPMS), di-t-butylmethylsilyl (DTBMS), acetate, chloroacetate, methoxyacetate, triphenylmethoxyacetate, pivaloate, benzoate, and p-toluenesulfonate (Ts)).

Since insensitivity of the functional group-protected DAG represented by Chemical Formula 1 is enhanced rather than DAG that is a sensitive compound, work may be performed safely from threats of explosion and fire accidents caused by impact, friction and static electricity, in comparison to a synthesis method according to the related art.

In one example embodiment, the functional group-protected DAG may have an impact sensitivity of 1.5 J to 19 J, a friction sensitivity of 5 N to 350 N, and an electrostatic sensitivity of 7 mJ to 50 mJ. The impact sensitivity, the friction sensitivity, and the electrostatic sensitivity are not limited to the above ranges, and the functional group-protected DAG may only need to be more insensitive than DAG (the DAG with an impact sensitivity of 1.5 J, a friction sensitivity of 5 N, and an electrostatic sensitivity of 7 mJ or greater).

In one example embodiment, the functional group-protected DAG may be synthesized from dichloroglyoxime (that may be hereinafter referred to as “DCG”).

In one example embodiment, the DCG may be synthesized from glyoxime.

In one example embodiment, the functional group-protected DAG may be synthesized from R-DCG that is synthesized from DCG and that is represented by the following Chemical Formula 2:

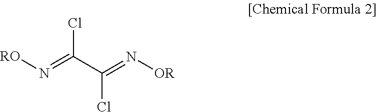

(Here, R includes at least one selected from the group consisting of tetrahydropyranyl (THP), methyl (Me), methoxymethyl (MOM), methoxythiomethyl (MTM), benzyloxymethyl (BOM), 2-methoxymethyl (MEM), 2-(trimethylsilyl)ethoxymethyl (SEM), tetrahydrofuranyl (THF), t-butyl, allyl, benzyl, p-methoxybenzyl, 3,4-dimethoxybenzyl, o-nitrobenzyl, p-nitrobenzyl, triphenylmethyl, trimethylsilyl (TMS), triethylsilyl (TES), triisopropylsilyl (TIPS), t-butyldimethylsilyl (TBDMS), t-butyldiphenylsilyl (TBDPS), diphenylmethylsilyl (DPMS), di-t-butylmethylsilyl (DTBMS), acetate, chloroacetate, methoxyacetate, triphenylmethoxyacetate, pivaloate, benzoate, and p-toluenesulfonate (Ts)).

In one example embodiment, the functional group-protected DAG may be an intermediate for preparation of one selected from the group consisting of an insensitive explosive, a non-toxic low-temperature gas generator, low-lead and/or lead-free pyrotechnics, and pharmaceutical chemicals.

In one example embodiment, the insensitive explosive may be dihydroxylammonium 5,5′-bistetrazole-1,1′-diolate (TKX-50).

According to another example embodiment of the present invention, a method of synthesizing functional group-protected DAG includes preparing DCG as a starting material; and forming R-DAG represented by the following Chemical Formula 1 from the DCG:

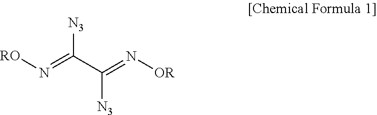

(Here, R includes at least one selected from the group consisting of tetrahydropyranyl (THP), methyl (Me), methoxymethyl (MOM), methoxythiomethyl (MTM), benzyloxymethyl (BOM), 2-methoxymethyl (MEM), 2-(trimethylsilyl)ethoxymethyl (SEM), tetrahydrofuranyl (THF), t-butyl, allyl, benzyl, p-methoxybenzyl, 3,4-dimethoxybenzyl, o-nitrobenzyl, p-nitrobenzyl, triphenylmethyl, trimethylsilyl (TMS), triethylsilyl (TES), triisopropylsilyl (TIPS), t-butyldimethylsilyl (TBDMS), t-butyldiphenylsilyl (TBDPS), diphenylmethylsilyl (DPMS), di-t-butylmethylsilyl (DTBMS), acetate, chloroacetate, methoxyacetate, triphenylmethoxyacetate, pivaloate, benzoate, and p-toluenesulfonate (Ts)).

In one example embodiment, the R-DAG may have an impact sensitivity of 1.5 J to 19 J, a friction sensitivity of 5 N to 350 N, and an electrostatic sensitivity of 7 mJ to 50 mJ. The impact sensitivity, the friction sensitivity, and the electrostatic sensitivity are not limited to the above ranges, and the R-DAG may only need to be more insensitive than DAG (the DAG with an impact sensitivity of 1.5 J, a friction sensitivity of 5 N, and an electrostatic sensitivity of 7 mJ or greater).

FIG. 2 is a diagram illustrating a method of synthesizing functional group-protected DAG according to an example embodiment of the present invention. As shown in FIG. 2, a process of synthesizing functional group-protected DAG according to an example embodiment of the present invention is described below.

In one example embodiment, the method may include synthesizing dichloroglyoxime (DCG); synthesizing R-DCG represented by the following Chemical Formula 2 through the DCG; and synthesizing R-DAG through the R-DCG:

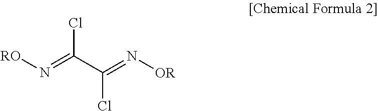

(Here, R includes at least one selected from the group consisting of tetrahydropyranyl (THP), methyl (Me), methoxymethyl (MOM), methoxythiomethyl (MTM), benzyloxymethyl (BOM), 2-methoxymethyl (MEM), 2-(trimethylsilyl)ethoxymethyl (SEM), tetrahydrofuranyl (THF), t-butyl, allyl, benzyl, p-methoxybenzyl, 3,4-dimethoxybenzyl, o-nitrobenzyl, p-nitrobenzyl, triphenylmethyl, trimethylsilyl (TMS), triethylsilyl (TES), triisopropylsilyl (TIPS), t-butyldimethylsilyl (TBDMS), t-butyldiphenylsilyl (TBDPS), diphenylmethylsilyl (DPMS), di-t-butylmethylsilyl (DTBMS), acetate, chloroacetate, methoxyacetate, triphenylmethoxyacetate, pivaloate, benzoate, and p-toluenesulfonate (Ts)).

In one example embodiment, the synthesizing of the dichloroglyoxime (DCG) may include synthesizing glyoxime; and reacting the glyoxime with N-chlorosuccinimide.

In the present invention, for example, in the synthesizing of the glyoxime, sodium hydroxide (NaOH) and distilled water may be added to a reactor, cooling may be performed to 0° C., hydroxylammonium chloride may be added to the reactor, and an aqueous glyoxal solution may be added to the reactor while maintaining a temperature of 0 to 10° C. Subsequently, when a solid is produced after stirring for a predetermined period of time while maintaining an internal temperature of the reactor at 0° C., the solid may be filtered, washed with a small amount of ice water, and then dried, to obtain glyoxime.

In one example embodiment, the synthesizing of the R-DCG through the DCG may be performed by reacting the DCG with a compound including at least one selected from the group consisting of tetrahydropyranyl (THP), methyl (Me), methoxymethyl (MOM), methoxythiomethyl (MTM), benzyloxymethyl (BOM), 2-methoxymethyl (MEM), 2-(trimethylsilyl)ethoxymethyl (SEM), tetrahydrofuranyl (THF), t-butyl, allyl, benzyl, p-methoxybenzyl, 3,4-dimethoxybenzyl, o-nitrobenzyl, p-nitrobenzyl, triphenylmethyl, trimethylsilyl (TMS), triethylsilyl (TES), triisopropylsilyl (TIPS), t-butyldimethylsilyl (TBDMS), t-butyldiphenylsilyl (TBDPS), diphenylmethylsilyl (DPMS), di-t-butylmethylsilyl (DTBMS), acetate, chloroacetate, methoxyacetate, triphenylmethoxyacetate, pivaloate, benzoate, and p-toluenesulfonate (Ts).

In one example embodiment, the synthesizing of the R-DCG through the DCG may be performed in the presence of a pyridinium p-toluenesulfonate (PPTS) catalyst. Although a catalyst is limited to the PPTS catalyst in the synthesizing of the R-DCG through the DCG, catalysts other than the PPTS catalyst may also be used.

In the present invention, for example, the synthesizing of the R-DCG through the DCG may be performed by reacting the DCG with 3,4-dihydro-2H-pyran (DHP) in the presence of the PPTS catalyst.

In one example embodiment, the synthesizing of the R-DCG through the DCG may be performed by stirring and reacting the DCG, the PPTS catalyst, and the compound at a molar ratio of 0.5 to 2:0.02 to 0.5:3 to 7. Desirably, the stirring and reacting may be performed at a molar ratio of 1:0.1:5. When the molar ratio is out of the above-described range, a yield may be reduced, or impurities may increase.

In one example embodiment, the stirring may be performed at a temperature of room temperature to 60° C. When the stirring is performed under a temperature condition outside the room temperature, a side reaction may occur. Desirably, the stirring may be performed at 50° C.

In the present invention, for example, synthesizing of O,O′-ditetrahydropyranyl oxalohydroximoyl dichloride (hereinafter, referred to as “THP-DCG”) as THP-DCG through the DCG may include 1) adding 2.98 g (18.98 mmol) of DCG, 35 mL of DCM, 0.498 g (1.98 mmol) of PPTS, and 8.298 g (98.65 mmol) of 3,4-dihydro-2H-pyran (DHP) to a reactor, followed by stirring at 50° C. for 3 hours, 2) adding 200 mL of diethyl ether, transferring a reaction solution to a separatory funnel and performing washing with 150 mL of saturated NaHCO₃ solution, 150 mL of saturated NaCl solution, and 150 mL of distilled water, and evaporating a solvent under reduced pressure to obtain THP-DCG.

In one example embodiment, the synthesizing of the R-DAG through the R-DCG may be performed through an azidation reaction.

In one example embodiment, the synthesizing of the R-DAG through the R-DCG may be performed by reacting the R-DCG with sodium azide (NaN₃).

In one example embodiment, the synthesizing of the R-DAG through the R-DCG may be performed by stirring and reacting the R-DCG and the sodium azide at a molar ratio of 1:2 to 4. Desirably, the stirring and reacting may be performed at a molar ratio of 1:3. When the molar ratio is out of the above-described range, a yield may be reduced, or impurities may increase.

In one example embodiment, the stirring may be performed at a temperature of 95° C. to 100° C. When the stirring is performed under a temperature condition outside the temperature of 95° C. to 100° C., the reaction may be less performed, which may result in a decrease in a yield or a side reaction.

In the present invention, for example, synthesizing of THP-DAG through the THP-DCG may include 1) adding 5 g (15.4 mmol) of THP-DCG, 100 mL of DMF and 3.0 g (46.2 mmol) of NaN₃, raising an internal temperature of a reactor to 100° C. and performing stirring for 2 hours, followed by cooling to room temperature, and 2) adding 100 mL of distilled water, precipitating THP-DAG and performing filtration to obtain THP-DAG.

By the method of synthesizing functional group-protected DAG according to an example embodiment of the present invention, R-DAG with enhanced insensitivity, instead of sensitive DAG, may be synthesized to reduce risks of processes and harmfulness arising from threats of explosion and fire accidents caused by impact, friction and static electricity, and thus the functional group-protected DAG may be stably synthesized. Also, it is possible to use the synthesized functional group-protected DAG as an intermediate to synthesize various materials.

According to another example embodiment of the present invention, a method of synthesizing TKX-50 using functional group-protected DAG includes preparing DCG as a starting material; forming an insensitive-DAG intermediate represented by the following Chemical Formula 1 from the DCG; and synthesizing TKX-50 through the insensitive-DAG intermediate:

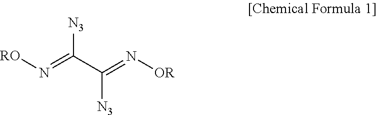

(Here, R includes at least one selected from the group consisting of tetrahydropyranyl (THP), methyl (Me), methoxymethyl (MOM), methoxythiomethyl (MTM), benzyloxymethyl (BOM), 2-methoxymethyl (MEM), 2-(trimethylsilyl)ethoxymethyl (SEM), tetrahydrofuranyl (THF), t-butyl, allyl, benzyl, p-methoxybenzyl, 3,4-dimethoxybenzyl, o-nitrobenzyl, p-nitrobenzyl, triphenylmethyl, trimethylsilyl (TMS), triethylsilyl (TES), triisopropylsilyl (TIPS), t-butyldimethylsilyl (TBDMS), t-butyldiphenylsilyl (TBDPS), diphenylmethylsilyl (DPMS), di-t-butylmethylsilyl (DTBMS), acetate, chloroacetate, methoxyacetate, triphenylmethoxyacetate, pivaloate, benzoate, and p-toluenesulfonate (Ts)).

Since insensitivity of the insensitive-DAG intermediate represented by Chemical Formula 1 is enhanced rather than DAG that is a sensitive intermediate synthesized during a synthesis of TKX-50, work may be performed safely from threats of explosion and fire accidents caused by impact, friction and static electricity using the insensitive-DAG intermediate, in comparison to a synthesis method according to the related art.

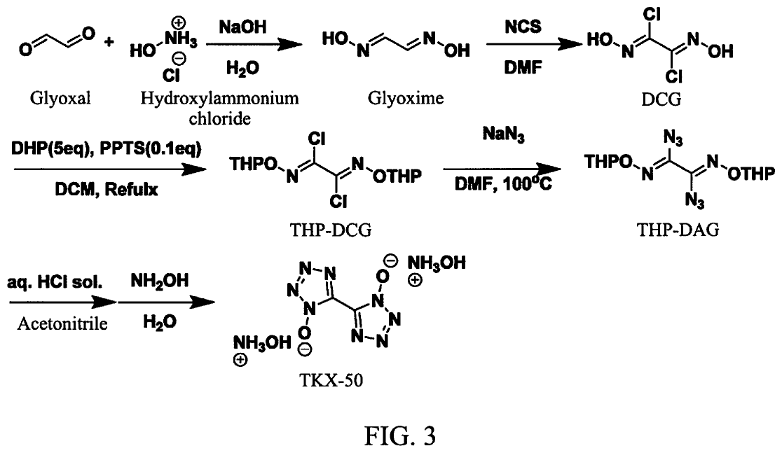

FIG. 3 is a diagram illustrating a method of synthesizing TKX-50 using functional group-protected DAG according to an example embodiment of the present invention. Hereinafter, a method of synthesizing TKX-50 using a THP-DAG intermediate as an insensitive-DAG intermediate according to an example embodiment of the present invention will be described with reference to FIG. 3.

The method of synthesizing TKX-50 using functional group-protected DAG according to an example embodiment of the present invention includes preparing dichloroglyoxime (that may be hereinafter referred to as “DCG”) as a starting material; forming O,O′-ditetrahydropyranyl oxalohydroximoyl diazide (hereinafter, referred to as “THP-DAG”) intermediate from the DCG; and synthesizing dihydroxylammonium 5,5′-bistetrazole-1,1′-diolate (hereinafter, referred to as “TKX-50”) through the THP-DAG intermediate.

In one example embodiment, the TKX-50 may be free of diazidoglyoxime (that may be hereinafter referred to as “DAG”) that is an intermediate byproduct.

In other words, a relatively insensitive insensitive-DAG intermediate may be used instead of DAG that is a sensitive intermediate during a synthesis of TKX-50, and thus an operator may more safely synthesize TKX-50.

In one example embodiment, the insensitive-DAG intermediate may have an impact sensitivity of 1.5 J to 19 J, a friction sensitivity of 5 N to 350 N, and an electrostatic sensitivity of 7 mJ to 50 mJ. The impact sensitivity, the friction sensitivity and the electrostatic sensitivity are not limited to the above-described ranges, and the insensitive-DAG intermediate may only need to be more insensitive than DAG (the DAG with an impact sensitivity of 1.5 J, a friction sensitivity of 5 N, and an electrostatic sensitivity of 7 mJ or greater).

Table 1 shows sensitivity characteristics of DAG and a THP-DAG intermediate that is an example of an insensitive-DAG intermediate. Specifically, after synthesis of THP-DAG, 5,5′-bistetrazole-1,1′-diol dihydrate (1,1′-BTO) was synthesized using a 37% HCl solution in an acetonitrile solvent, the solvent was evaporated out, and a one-pot reaction with hydroxylamine was performed, to finally synthesize dihydroxylammonium 5,5′-bistetrazole-1,1′-diolate (TKX-50). More specifically, results obtained by measuring the impact sensitivity, the friction sensitivity and the electrostatic sensitivity of the THP-DAG using a BAM Fall Hammer, a BAM Friction Tester, and an Electrostatic Spark Sensitivity Tester are shown.

TABLE 1

| | Impact | Friction | Electrostatic |
|---|---|---|---|
| | sensitivity | sensitivity | sensitivity |
| | [J] | [N] | [mJ] |
| DAG | 1.5 | <5 | 7 |
| THP-DAG | 19.95 | 352.8 | 50 |

Referring to Table 1, it is confirmed that THP-DAG having an impact sensitivity of 19.95 J, a friction sensitivity of 352.8 N and an electrostatic sensitivity of 50 mJ is much more insensitive than the DAG. In particular, referring to the impact sensitivity/friction sensitivity of the THP-DAG, the THP-DAG is much more insensitive than a high-energy material that is already in use. It is possible to perform work safely from threats of explosion and fire accidents caused by impact/friction/static electricity in handling of the THP-DAG, in comparison to using existing synthesis methods. In one example embodiment, the method may include synthesizing dichloroglyoxime (DCG); synthesizing an R-DCG intermediate represented by the following Chemical Formula 2 through the DCG; synthesizing an insensitive-DAG intermediate through the R-DCG intermediate; and synthesizing TKX-50 through the insensitive-DAG intermediate:

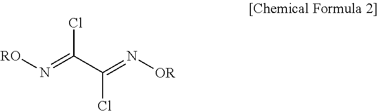

(Here, R includes at least one selected from the group consisting of tetrahydropyranyl (THP), methyl (Me), methoxymethyl (MOM), methoxythiomethyl (MTM), benzyloxymethyl (BOM), 2-methoxymethyl (MEM), 2-(trimethylsilyl)ethoxymethyl (SEM), tetrahydrofuranyl (THF), t-butyl, allyl, benzyl, p-methoxybenzyl, 3,4-dimethoxybenzyl, o-nitrobenzyl, p-nitrobenzyl, triphenylmethyl, trimethylsilyl (TMS), triethylsilyl (TES), triisopropylsilyl (TIPS), t-butyldimethylsilyl (TBDMS), t-butyldiphenylsilyl (TBDPS), diphenylmethylsilyl (DPMS), di-t-butylmethylsilyl (DTBMS), acetate, chloroacetate, methoxyacetate, triphenylmethoxyacetate, pivaloate, benzoate, and p-toluenesulfonate (Ts)).

In one example embodiment, the method may include, when an THP-DCG intermediate and a THP-DAG intermediate are used, synthesizing dichloroglyoxime (DCG); synthesizing O,O′-ditetrahydropyranyl oxalohydroximoyl dichloride (hereinafter, referred to as “THP-DCG”) through the DCG; synthesizing THP-DAG through the THP-DCG; and synthesizing TKX-50 through the THP-DAG.

In one example embodiment, the synthesizing of the dichloroglyoxime (DCG) may include synthesizing glyoxime; and reacting the glyoxime with N-chlorosuccinimide.

In one example embodiment, the synthesizing of the R-DCG intermediate through the DCG may be performed by reacting the DCG with a compound including at least one selected from the group consisting of tetrahydropyranyl (THP), methyl (Me), methoxymethyl (MOM), methoxythiomethyl (MTM), benzyloxymethyl (BOM), 2-methoxymethyl (MEM), 2-(trimethylsilyl)ethoxymethyl (SEM), tetrahydrofuranyl (THF), t-butyl, allyl, benzyl, p-methoxybenzyl, 3,4-dimethoxybenzyl, o-nitrobenzyl, p-nitrobenzyl, triphenylmethyl, trimethylsilyl (TMS), triethylsilyl (TES), triisopropylsilyl (TIPS), t-butyldimethylsilyl (TBDMS), t-butyldiphenylsilyl (TBDPS), diphenylmethylsilyl (DPMS), di-t-butylmethylsilyl (DTBMS), acetate, chloroacetate, methoxyacetate, triphenylmethoxyacetate, pivaloate, benzoate, and p-toluenesulfonate (Ts).

In one example embodiment, the synthesizing of the R-DCG intermediate through the DCG may be performed in the presence of a pyridinium p-toluenesulfonate (PPTS) catalyst. Although a catalyst is limited to the PPTS catalyst in the synthesizing of the R-DCG through the DCG, catalysts other than the PPTS catalyst may also be used.

In the present invention, for example, the synthesizing of the R-DCG intermediate through the DCG may be performed by reacting the DCG with 3,4-dihydro-2H-pyran (DHP) in the presence of the PPTS catalyst.

In one example embodiment, the synthesizing of the R-DCG intermediate through the DCG may be performed by stirring and reacting the DCG, the PPTS catalyst, and the compound at a molar ratio of 1:0.1:5. Desirably, the stirring and reacting may be performed at a molar ratio of 1:0.1:5.

When the molar ratio is out of the above-described range, a yield may be reduced, or impurities may increase.

In one example embodiment, the stirring of the DCG, the PPTS catalyst, and the compound may be performed at a temperature of room temperature to 60° C. When the stirring is performed under a temperature condition outside the room temperature, a side reaction may occur. Desirably, the stirring may be performed at 50° C.

As an example, the synthesizing of the THP-DCG through the DCG may include adding 2.98 g (18.98 mmol) of DCG, 35 mL of DCM, 0.498 g (1.98 mmol) of PPTS, and 8.298 g (98.65 mmol) of 3,4-dihydro-2H-pyran (DHP) to a reactor, followed by stirring at 50° C. for 3 hours, 2) adding 200 mL of diethyl ether, transferring a reaction solution to a separatory funnel and performing washing with 150 mL of saturated NaHCO₃ solution, 150 mL of saturated NaCl solution, and 150 mL of distilled water, and evaporating a solvent under reduced pressure to obtain THP-DCG.

In one example embodiment, the synthesizing of the insensitive-DAG intermediate through the R-DCG intermediate may be performed through an azidation reaction.

In one example embodiment, the synthesizing of the insensitive-DAG intermediate through the R-DCG intermediate may be performed by reacting the R-DCG intermediate with sodium azide (NaN₃).

In one example embodiment, the R-DCG intermediate and the sodium azide may be stirred at a molar ratio of 1:2 to 4 and reacted. Desirably, the R-DCG intermediate and the sodium azide may be stirred at a molar ratio of 1:3 and reacted.

When the molar ratio is out of the above-described range, a yield may be reduced, or impurities may increase.

In one example embodiment, stirring of the R-DCG intermediate and the sodium azide may be performed at a temperature of 95° C. to 100° C. When the stirring is performed under a temperature condition outside the temperature of 95° C. to 100° C., the reaction may be less performed, which may result in a decrease in a yield or a side reaction.

As an example, the synthesizing of the THP-DAG through the THP-DCG may include 1) adding 5 g (15.4 mmol) of THP-DCG, 100 mL of DMF and 3.0 g (46.2 mmol) of NaN₃, raising an internal temperature of a reactor to 100° C. and performing stirring for 2 hours, followed by cooling to room temperature, and 2) adding 100 mL of distilled water, precipitating THP-DAG and performing filtration, to obtain THP-DAG.

In one example embodiment, the synthesizing of the TKX-50 through the insensitive-DAG intermediate may include synthesizing 5,5′-bistetrazole-1,1′-diol by reacting the insensitive-DAG intermediate with an aqueous hydrochloric acid solution; and synthesizing TKX-50 by reacting the 5,5′-bistetrazole-1,1′-diol with hydroxylamine.

In an example, when the insensitive-DAG intermediate is reacted in an aqueous acid solution, 5,5′-bistetrazole-1,1′-diol without an R group may be obtained. In another example, 5-5′-bistetrazole-1,1′-protected diol with an R group may be obtained. In the case of the 5-5′-bistetrazole-1,1′-protected diol with the R group, a reaction of removing the R group may be added.

In one example embodiment, the synthesizing of the 5,5′-bistetrazole-1,1′-diol (1,1′-BTO) by reacting the insensitive-DAG intermediate with the aqueous hydrochloric acid solution may be performed by stirring the insensitive-DAG intermediate and the aqueous hydrochloric acid solution under a temperature condition of room temperature.

As an example, the synthesizing of the 5,5′-bistetrazole-1,1′-diol by reacting THP-DAG with the aqueous hydrochloric acid solution may include 1) adding 0.5 g (1.47 mmol) of THP-DAG and 50 mL of acetonitrile to a reactor at room temperature, and injecting 1.0 mL (12.1 mmol) of a 37% HCl solution, 2) sealing the reactor and performing stirring at room temperature for 24 hours, and 3) precipitating 1,1′-BTO by removing the acetonitrile and the HCl solution by blowing with air.

In one example embodiment, the synthesizing of the TKX-50 by reacting the 5,5′-bistetrazole-1,1′-diol with the hydroxylamine may be performed by stirring and reacting the 5,5′-bistetrazole-1,1′-diol and the hydroxylamine at a molar ratio of 1:3 to 50. Desirably, the stirring and reacting may be performed at a molar ratio of 1:44.

When the molar ratio is out of the above-described range, a yield may be reduced, or impurities may increase.

In one example embodiment, the stirring of the 5,5′-bistetrazole-1,1′-diol and the hydroxylamine may be performed at a temperature of 40° C. to 60° C. Desirably, the stirring may be performed at 50° C. When the stirring is performed under a temperature condition outside the temperature of 40° C. to 60° C., a reaction yield may be reduced, or impurities may increase.

As an example, the synthesizing of the TKX-50 by reacting the 5,5′-bistetrazole-1,1′-diol with the hydroxylamine may include 1) adding 10 mL of distilled water to a reactor containing 1,1′-BTO, raising the internal temperature of the reactor to 50° C. and adding 4.0 mL (65.3 mmol) of NH₂OH (50% w/w in H₂O), and 2) performing stirring at 50° C. for 30 minutes and cooling the reactor to room temperature to precipitate TKX-50, followed by filtration and drying, to obtain the TKX-50.

According to an example embodiment of the present invention, by a method of synthesizing TKX-50 using functional group-protected DAG, it is possible to perform work safely from threats of explosion and fire accidents caused by impact, friction and static electricity through an insensitive-DAG intermediate that is an intermediate with enhanced insensitivity, instead of DAG that is a sensitive intermediate synthesized during a synthesis of TKX-50, in comparison to a synthesis method according to a related art. Also, by using an aqueous HCl solution instead of HCl gas, it is possible to easily and safely perform a process.

Hereinafter, the present invention will be described in more detail with reference to examples and comparative examples.

However, the following examples are given to illustrate the present invention, and the present invention is not limited to the examples.

### EXAMPLES

Analysis of Materials and Characteristics

All chemicals were received as pure analytical grade materials obtained from Acros or Aldrich Organics. ¹H and ¹³C NMR spectra were found in a 400 MHz nuclear magnetic resonance (NMR) spectrometer (Bruker AVANCE 400) that uses DMSO-d₆ or CDCl₃ as a solvent. Analytical thin layer chromatography (TLC) was performed with E. Merck precoated TLC plates, layer thickness 0.25 mm and silica gel 60F-254. High-resolution mass spectra were obtained from a microTOE-QII HRMS/MS instrument (Bruker) with electrospray ionization technology. Impact sensitivity tests were carried out using a BAM Fall hammer instrument (OZM) according to STANAG 4489 modified instruction. Friction sensitivity tests were carried out using a BAM friction tester (OZM) according to STANAG 4487 modified instruction. Electrostatic discharge tests were carried out using an ESD tester (OZM) according to STANAG 4490.

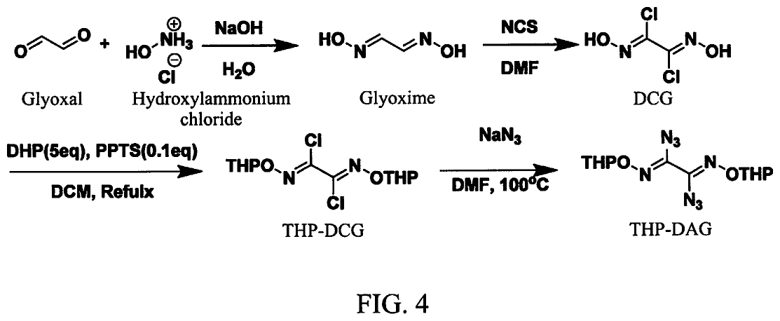

FIG. 4 is a diagram illustrating a synthesis method of THP-DAG synthesized in Examples 1 to 4 according to the present invention. As shown in FIG. 4, a synthesis of

THP-DAG as functional group-protected DAG according to an example embodiment of the present invention will be described below in Examples 1 to 4.

### Example 1: Synthesis of Glyoxime

18.4 g (0.46 mol) of NaOH and 50 mL of distilled water were added to a reactor, cooled to 0° C., and 46 g (0.66 mol) of hydroxylammonium chloride was added to the reactor. Subsequently, 47.9 g (0.33 mol) of 40% aqueous glyoxal solution was added to the reactor while maintaining a temperature of 0 to 10° C. When a solid is produced after stirring for 1 hour while maintaining an internal temperature of the reactor at 0° C., the solid was filtered and washed with a small amount of ice water. Subsequently, drying was performed to obtain 24.7 g (0.28 mol, 85%) of glyoxime.

¹H NMR (DMSO-d₆): 7.73 (s, 2H, CH), 11.61 (s, 2H, OH); ¹³C NMR (DMSO-d₆): 145.82

### Example 2: Synthesis of DCG Through Glyoxime

18 g (0.20 mol) of glyoxime and 180 mL of DMF were added to the reactor, cooled to 0° C., and 54.5 g (0.40 mol) of N-chlorosuccinimide (NCS) was slowly added to the reactor. Subsequently, stirring was performed for 30 minutes while maintaining the internal temperature of the reactor at 0° C., the internal temperature was slowly raised to 25° C., and stirring was performed for 1 hour. Subsequently, after 200 mL of distilled water was added, a reaction solution was transferred to a separatory funnel and extraction was performed with 200 mL of EA and 150 mL of distilled water three times. After evaporating the obtained organic layer under reduced pressure, crude DCG was obtained. The obtained crude DCG and 100 mL of MC were added to the reactor and stirred at room temperature for 1 hour, followed by filtration. Subsequently, drying was performed to obtain 25.4 g (0.16 mol, 81%) of DCG.

¹H NMR (DMSO-d₆): 13.10 (s, 2H, OH); ¹³C NMR (DMSO-d₆): 130.86

### Example 3: Synthesis of THP-DCG Through DCG

2.98 g (18.98 mmol) of DCG, 35 mL of DCM, 0.498 g (1.98 mmol) of PPTS, and 8.298 g (98.65 mmol) of 3,4-dihydro-2H-pyran (DHP) were added to the reactor and stirred at room temperature for 3 hours. Subsequently, after 200 mL of diethyl ether was added, a reaction solution was transferred to a separatory funnel, and washing with 150 mL of saturated NaHCO₃ solution, 150 mL of saturated NaCl solution, and 150 mL of distilled water was performed. Subsequently, a solvent was evaporated under reduced pressure, to obtain 4.34 g (13.28 mmol, 70%) of THP-DCG.

¹H NMR (CDCl₃): 1.64 (m, 8H, CH₂), 1.86 (m, 4H, CH₂), 3.75 (m, 4H, CH₂), 5.52 (m, 2H, CH); ¹³C NMR (CDCl₃): 18.80, 18.83, 25.16, 28.45, 28.47, 62.54, 62.62, 102.30, 102.36, 133.91, 133.96

### Example 4: Synthesis of THP-DAG Through THP-DCG

5 g (15.4 mmol) of THP-DCG, 100 mL of DMF, and 3.0 g (46.2 mmol) of NaN₃ were added to the reactor. The internal temperature of the reactor was raised to 100° C. and stirring was performed for 2 hours, followed by cooling to room temperature. Subsequently, 100 mL of distilled water was added, and THP-DAG was precipitated and filtered, to obtain 4.11 g (12.166 mmol, 79%) of THP-DAG.

¹H NMR (CDCl₃): 1.63 (m, 8H, CH₂), 1.80 (m, 4H, CH₂), 3.75 (m, 4H, CH₂), 5.34 (m, 2H, CH); ¹³C NMR (CDCl₃): 18.37, 18.45, 24.79, 28.01, 28.06, 62.10, 62.26, 101.72, 101.81, 137.80, 137.82; impact sensitivity: 19.95 J, friction sensitivity: 352.8 N, electrostatic sensitivity: 50 mJ

### Example 5: Synthesis of 1,1′-BTO Through THP-DAG and Synthesis of TKX-50 Through Synthesis of 1,1′-BTO

0.5 g (1.47 mmol) of THP-DAG and 50 mL of acetonitrile were added to the reactor at room temperature, and 1.0 mL (12.1 mmol) of a 37% HCl solution was then added thereto. Subsequently, the reactor was sealed and stirring was performed at room temperature for 24 hours. After the reaction, the acetonitrile and HCl solution were removed by blowing with air to precipitate 1,1′-BTO. 10 mL of distilled water was added to the reactor containing the precipitated 1,1′-BTO, and the internal temperature of the reactor was raised to 50° C., to melt the 1,1′-BTO. After 4.0 mL (65.3 mmol) of NH₂OH (50% w/w in H₂O) was added, stirring was performed for 2 hours while slowly cooling to room temperature. Precipitated TKX-50 was filtered and dried, to obtain 0.22 g (0.931 mmol, 63.3% in two steps) of TKX-50.

¹H NMR (DMSO-d₆): 9.74 (s, 8H, NH₃OH); ¹³C NMR (DMSO-d₆): 135.48

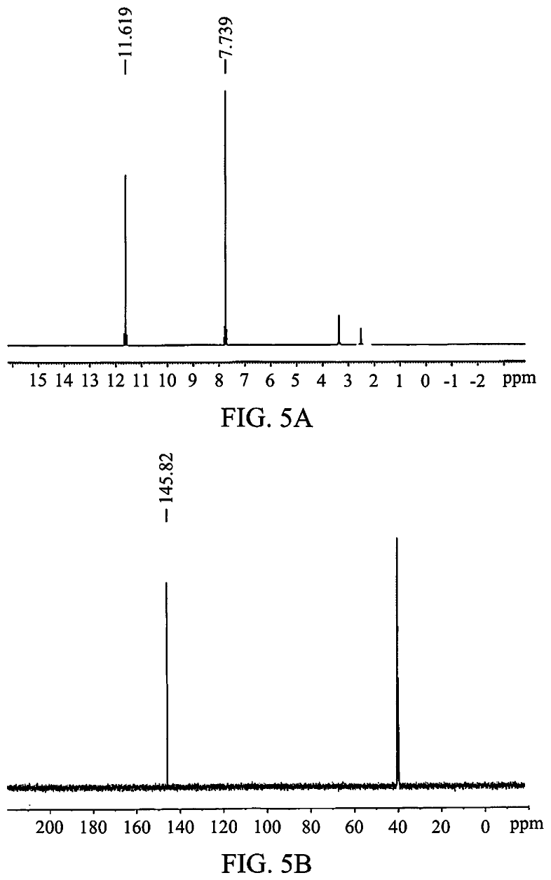

Referring to FIGS. 5A and 5B, it may be found that the glyoxime was synthesized based on Example 1.

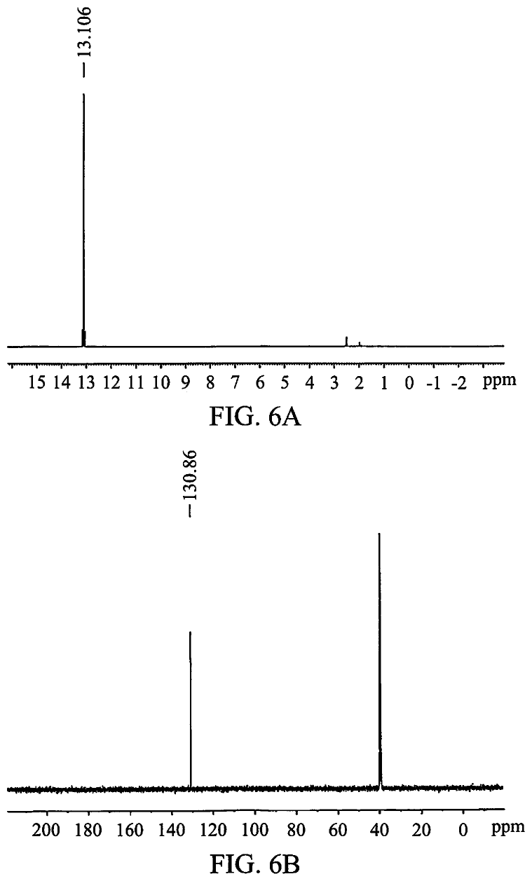

FIG. 6 illustrates NMR graphs of the DCG synthesized in Example 2 of the present invention. More specifically, FIG. 6A is a′1-1 NMR spectrum of the DCG and FIG. 6B is a ¹³C NMR spectrum of the DCG.

Referring to FIGS. 6A and 6B, it may be found that the DCG was synthesized based on Example 2.

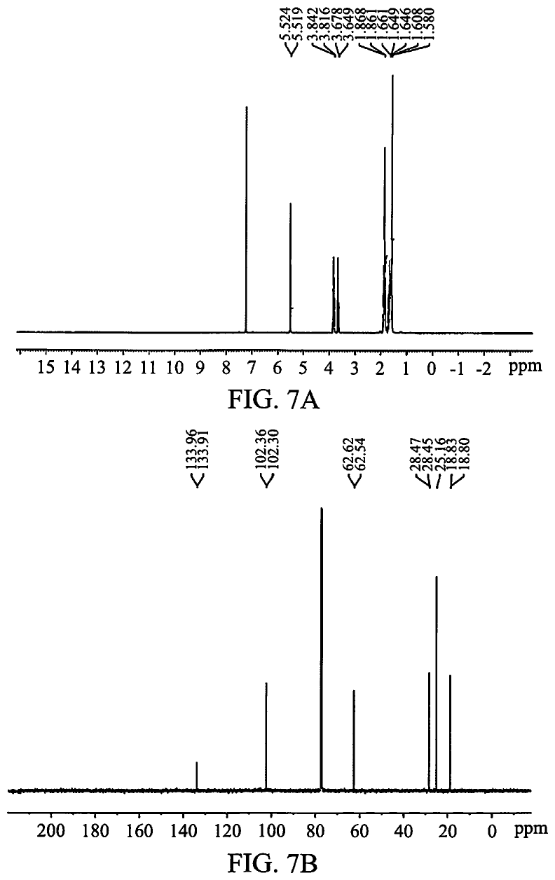

FIG. 7 illustrates NMR graphs of the THP-DCG synthesized in Example 3 of the present invention. More specifically, FIG. 7A is a ¹H NMR spectrum of the THP-DCG and FIG. 7B is a ¹³C NMR spectrum of the THP-DCG.

Referring to FIGS. 7A and 7B, it may be found that the THP-DCG was synthesized based on Example 3.

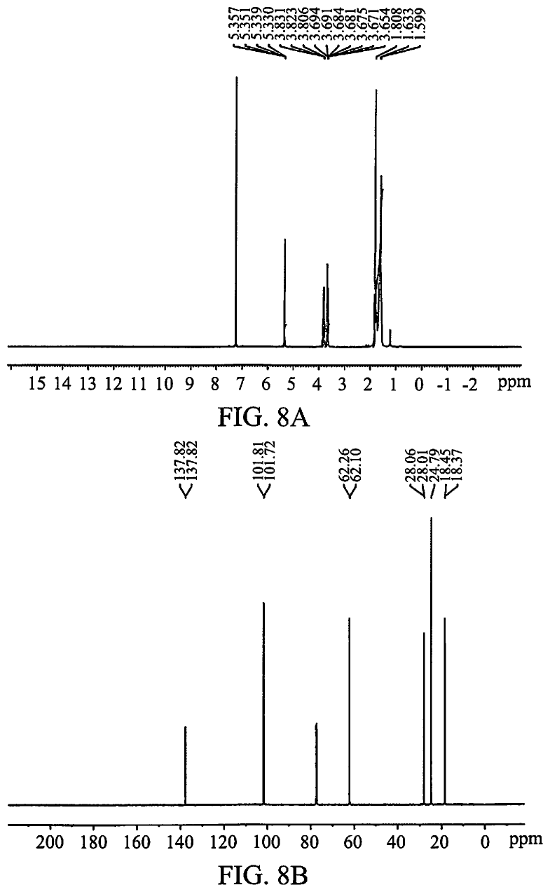

FIG. 8 illustrates NMR graphs of the TI-IP-DAG synthesized in Example 4 of the present invention. More specifically, FIG. 8A is a ¹H NMR spectrum of the THP-DAG and FIG. 8B is a ¹³C NMR spectrum of the THP-DAG.

Referring to FIGS. 8A and 8B, it may be found that the THP-DAG was synthesized based on Example 4.

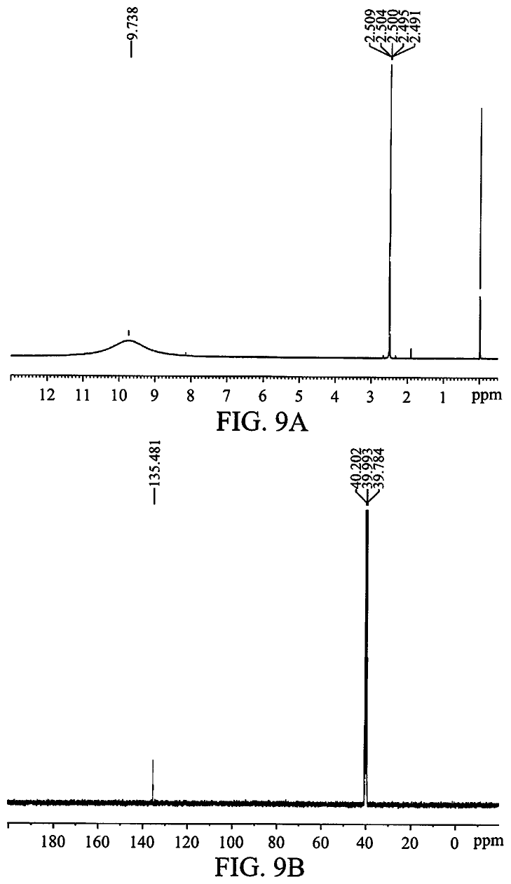

FIG. 9 illustrates NMR graphs of the TKX-50 synthesized in Example 5 of the present invention. More specifically, FIG. 9A is a ¹H NMR spectrum of the TKX-50 and FIG. 9B is a ¹³C NMR spectrum of the TKX-50.

Referring to FIGS. 9A and 9B, it may be found that the TKX-50 was synthesized based on Example 5. As described above, the present invention relates to a method of synthesizing TKX-50 through THP-DAG that is an intermediate with enhanced insensitivity, instead of DAG that is a sensitive intermediate synthesized during a synthesis of TKX-50, and is advantageous in that work may be safely performed from threats of explosion and fire accidents caused by impact, friction and static electricity, in comparison to existing synthesis methods.

While the example embodiments have been shown and described with reference to the accompanying drawings, it will be apparent to those skilled in the art that various modifications and variations can be made from the foregoing descriptions. For example, adequate effects may be achieved even if the foregoing processes and methods are carried out in different order than described above, and/or the aforementioned elements are combined or coupled in different forms and modes than as described above or be substituted or switched with other components or equivalents. Thus, other implementations, alternative embodiments and equivalents to the claimed subject matter are construed as being within the appended claims.

1. Functional group-protected diazidoglyoxime (DAG) represented by the following Chemical Formula 1: 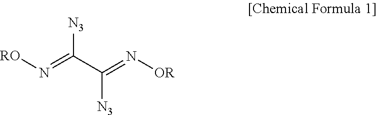
in which R includes at least one selected from the group consisting of tetrahydropyranyl (THP), methyl (Me), methoxymethyl (MOM), methoxythiomethyl (MTM), benzyloxymethyl (BOM), 2-methoxymethyl (MEM), 2-(trimethylsilyl)ethoxymethyl (SEM), tetrahydrofuranyl (THF), t-butyl, allyl, benzyl, p-methoxybenzyl, 3,4-dimethoxybenzyl, o-nitrobenzyl, p-nitrobenzyl, triphenylmethyl, trimethylsilyl (TMS), triethylsilyl (TES), triisopropylsilyl (TIPS), t-butyldimethylsilyl (TBDMS), t-butyldiphenylsilyl (TBDPS), diphenylmethylsilyl (DPMS), di-t-butylmethylsilyl (DTBMS), acetate, chloroacetate, methoxyacetate, triphenylmethoxyacetate, pivaloate, benzoate, and p-toluenesulfonate (Ts).

2. The functional group-protected DAG of claim 1, wherein the functional group-protected DAG has an impact sensitivity of 1.5 J to 19 J, a friction sensitivity of 5 N to 350 N, and an electrostatic sensitivity of 7 mJ to 50 mJ.

3. The functional group-protected DAG of claim 1, wherein
the functional group-protected DAG is synthesized from dichloroglyoxime (DCG), and
the functional group-protected DAG is synthesized from R-DCG that is synthesized from DCG and that is represented by the following Chemical Formula 2: 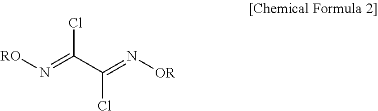
in which R includes at least one selected from the group consisting of tetrahydropyranyl (THP), methyl (Me), methoxymethyl (MOM), methoxythiomethyl (MTM), benzyloxymethyl (BOM), 2-methoxymethyl (MEM), 2-(trimethylsilyl)ethoxymethyl (SEM), tetrahydrofuranyl (THF), t-butyl, allyl, benzyl, p-methoxybenzyl, 3,4-dimethoxybenzyl, o-nitrobenzyl, p-nitrobenzyl, triphenylmethyl, trimethylsilyl (TMS), triethylsilyl (TES), triisopropylsilyl (TIPS), t-butyldimethylsilyl (TBDMS), t-butyldiphenylsilyl (TBDPS), diphenylmethylsilyl (DPMS), di-t-butylmethylsilyl (DTBMS), acetate, chloroacetate, methoxyacetate, triphenylmethoxyacetate, pivaloate, benzoate, and p-toluenesulfonate (Ts).

4. The functional group-protected DAG of claim 1, wherein
the functional group-protected DAG is an intermediate for preparation of one selected from the group consisting of an insensitive explosive, a non-toxic low-temperature gas generator, low-lead and/or lead-free pyrotechnics, and pharmaceutical chemicals, and
the insensitive explosive is dihydroxylammonium 5,5′-bistetrazole-1,1′-diolate (TKX-50).

5. A method of synthesizing functional group-protected diazidoglyoxime (DAG), the method comprising:
preparing dichloroglyoxime (DCG) as a starting material; and
forming R-DAG from the DCG, the R-DAG being represented by the following Chemical Formula 1:

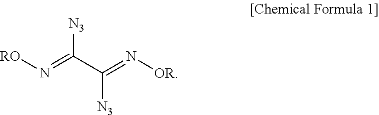

6. The method of claim 5, wherein the R-DAG has an impact sensitivity of 1.5 J to 19 J, a friction sensitivity of 5 N to 350 N, and an electrostatic sensitivity of 7 mJ to 50 mJ.

7. The method of claim 5, wherein the method comprises:
synthesizing dichloroglyoxime (DCG);
synthesizing R-DCG through the DCG, the R-DCG being represented by the following Chemical Formula 2; and
synthesizing R-DAG through the R-DCG: 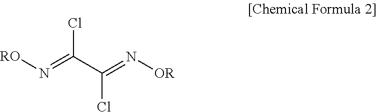
in which R includes at least one selected from the group consisting of tetrahydropyranyl (THP), methyl (Me), methoxymethyl (MOM), methoxythiomethyl (MTM), benzyloxymethyl (BOM), 2-methoxymethyl (MEM), 2-(trimethylsilyl)ethoxymethyl (SEM), tetrahydrofuranyl (THF), t-butyl, allyl, benzyl, p-methoxybenzyl, 3,4-dimethoxybenzyl, o-nitrobenzyl, p-nitrobenzyl, triphenylmethyl, trimethylsilyl (TMS), triethylsilyl (TES), triisopropylsilyl (TIPS), t-butyldimethylsilyl (TBDMS), t-butyldiphenylsilyl (TBDPS), diphenylmethylsilyl (DPMS), di-t-butylmethylsilyl (DTBMS), acetate, chloroacetate, methoxyacetate, triphenylmethoxyacetate, pivaloate, benzoate, and p-toluenesulfonate (Ts).

8. The method of claim 7, wherein the synthesizing of the dichloroglyoxime (DCG) comprises:
synthesizing glyoxime; and
reacting the glyoxime with N-chlorosuccinimide.

9. The method of claim 7, wherein
the synthesizing of the R-DCG through the DCG is performed by reacting the DCG with a compound including at least one selected from the group consisting of tetrahydropyranyl (THP), methyl (Me), methoxymethyl (MOM), methoxythiomethyl (MTM), benzyloxymethyl (BOM), 2-methoxymethyl (MEM), 2-(trimethylsilyl)ethoxymethyl (SEM), tetrahydrofuranyl (THF), t-butyl, allyl, benzyl, p-methoxybenzyl, 3,4-dimethoxybenzyl, o-nitrobenzyl, p-nitrobenzyl, triphenylmethyl, trimethylsilyl (TMS), triethylsilyl (TES), triisopropylsilyl (TIPS), t-butyldimethylsilyl (TBDMS), t-butyldiphenylsilyl (TBDPS), diphenylmethylsilyl (DPMS), di-t-butylmethylsilyl (DTBMS), acetate, chloroacetate, methoxyacetate, triphenylmethoxyacetate, pivaloate, benzoate, and p-toluenesulfonate (Ts),
the synthesizing of the R-DCG through the DCG is performed in the presence of a pyridinium p-toluenesulfonate (PPTS) catalyst,
the synthesizing of the R-DCG through the DCG is performed by stirring and reacting the DCG, the PPTS catalyst, and the compound at a molar ratio of 0.5 to 2:0.02 to 0.5:3 to 7, and
the stirring is performed at a temperature of room temperature to 60° C.

10. The method of claim 7, wherein
the synthesizing of the R-DAG through the R-DCG is performed through an azidation reaction,
the synthesizing of the R-DAG through the R-DCG is performed by reacting the R-DCG with sodium azide (NaN₃),
the synthesizing of the R-DAG through the R-DCG is performed by stirring and reacting the R-DCG and the sodium azide at a molar ratio of 1:2 to 4, and
the stirring is performed at a temperature of 95° C. to 100° C.

11. A method of synthesizing TKX-50 using functional group-protected diazidoglyoxime (DAG), the method comprising:
preparing dichloroglyoxime (DCG) as a starting material;
forming an insensitive-DAG intermediate from the DCG, the insensitive-DAG intermediate being represented by the following Chemical Formula 1; and
synthesizing TKX-50 through the insensitive-DAG intermediate: 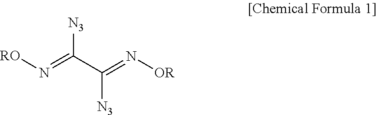
in which R includes at least one selected from the group consisting of tetrahydropyranyl (THP), methyl (Me), methoxymethyl (MOM), methoxythiomethyl (MTM), benzyloxymethyl (BOM), 2-methoxymethyl (MEM), 2-(trimethylsilyl)ethoxymethyl (SEM), tetrahydrofuranyl (THF), t-butyl, allyl, benzyl, p-methoxybenzyl, 3,4-dimethoxybenzyl, o-nitrobenzyl, p-nitrobenzyl, triphenylmethyl, trimethylsilyl (TMS), triethylsilyl (TES), triisopropylsilyl (TIPS), t-butyldimethylsilyl (TBDMS), t-butyldiphenylsilyl (TBDPS), diphenylmethylsilyl (DPMS), di-t-butylmethylsilyl (DTBMS), acetate, chloroacetate, methoxyacetate, triphenylmethoxyacetate, pivaloate, benzoate, and p-toluenesulfonate (Ts).

12. The method of claim 11, wherein the TKX-50 is free of diazidoglyoxime (DAG) that is an intermediate byproduct.

13. The method of claim 11, wherein the insensitive-DAG intermediate has an impact sensitivity of 1.5 J to 19 J, a friction sensitivity of 5 N to 300 N, and an electrostatic sensitivity of 7 mJ to 50 mJ

14. The method of claim 11, wherein the method comprises:
synthesizing dichloroglyoxime (DCG);
synthesizing an R-DCG intermediate through the DCG, the R-DCG intermediate being represented by the following Chemical Formula 2;
synthesizing an insensitive-DAG intermediate through the R-DCG intermediate; and
synthesizing TKX-50 through the insensitive-DAG intermediate: 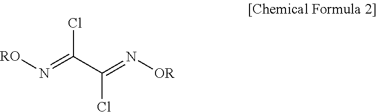
in which R includes at least one selected from the group consisting of tetrahydropyranyl (THP), methyl (Me), methoxymethyl (MOM), methoxythiomethyl (MTM), benzyloxymethyl (BOM), 2-methoxymethyl (MEM), 2-(trimethylsilyl)ethoxymethyl (SEM), tetrahydrofuranyl (THF), t-butyl, allyl, benzyl, p-methoxybenzyl, 3,4-dimethoxybenzyl, o-nitrobenzyl, p-nitrobenzyl, triphenylmethyl, trimethylsilyl (TMS), triethylsilyl (TES), triisopropylsilyl (TIPS), t-butyldimethylsilyl (TBDMS), t-butyldiphenylsilyl (TBDPS), diphenylmethylsilyl (DPMS), di-t-butylmethylsilyl (DTBMS), acetate, chloroacetate, methoxyacetate, triphenylmethoxyacetate, pivaloate, benzoate, and p-toluenesulfonate (Ts).

15. The method of claim 14, wherein the synthesizing of the dichloroglyoxime (DCG) comprises:
synthesizing glyoxime; and
reacting the glyoxime with N-chlorosuccinimide.

16. The method of claim 14, wherein the synthesizing of the R-DCG intermediate through the DCG is performed by reacting the DCG with a compound including at least one selected from the group consisting of tetrahydropyranyl (THP), methyl (Me), methoxymethyl (MOM), methoxythiomethyl (MTM), benzyloxymethyl (BOM), 2-methoxymethyl (MEM), 2-(trimethylsilyl)ethoxymethyl (SEM), tetrahydrofuranyl (THF), t-butyl, allyl, benzyl, p-methoxybenzyl, 3,4-dimethoxybenzyl, o-nitrobenzyl, p-nitrobenzyl, triphenylmethyl, trimethylsilyl (TMS), triethylsilyl (TES), triisopropylsilyl (TIPS), t-butyldimethylsilyl (TBDMS), t-butyldiphenylsilyl (TBDPS), diphenylmethylsilyl (DPMS), di-t-butylmethylsilyl (DTBMS), acetate, chloroacetate, methoxyacetate, triphenylmethoxyacetate, pivaloate, benzoate, and p-toluenesulfonate (Ts).

17. The method of claim 16, wherein
the synthesizing of the R-DCG intermediate through the DCG is performed in the presence of a pyridinium p-toluenesulfonate (PPTS) catalyst,
the synthesizing of the R-DCG intermediate through the DCG is performed by stirring and reacting the DCG, the PPTS catalyst, and the compound at a molar ratio of 1:0.1:5, and
the stirring of the DCG, the PPTS catalyst, and the compound is performed at a temperature of room temperature to 60° C.

18. The method of claim 14, wherein
the synthesizing of the insensitive-DAG intermediate through the R-DCG intermediate is performed through an azidation reaction,
the synthesizing of the insensitive-DAG intermediate through the R-DCG intermediate is performed by reacting the R-DCG intermediate with sodium azide (NaN₃),
the synthesizing of the insensitive-DAG intermediate through the R-DCG intermediate is performed by stirring and reacting the R-DCG intermediate and the sodium azide are stirred at a molar ratio of 1:2 to 4, and
the stirring of the R-DCG intermediate and the sodium azide is performed at a temperature of 95° C. to 100° C.

19. The method of claim 14, wherein the synthesizing of the TKX-50 through the insensitive-DAG intermediate comprises:
synthesizing 5,5′-bistetrazole-1,1′-diol by reacting the insensitive-DAG intermediate with an aqueous hydrochloric acid solution; and
synthesizing the TKX-50 by reacting the 5,5′-bistetrazole-1,1′-diol with hydroxylamine,
wherein the synthesizing of the 5,5′-bistetrazole-1,1′-diol by reacting the insensitive-DAG intermediate with the aqueous hydrochloric acid solution is performed by stirring the insensitive-DAG intermediate and the aqueous hydrochloric acid solution under a temperature condition of room temperature.

20. The method of claim 19, wherein
the synthesizing of the TKX-50 by reacting the 5,5′-bistetrazole-1,1′-diol with the hydroxylamine is performed by stirring and reacting the 5,5′-bistetrazole-1,1′-diol and the hydroxylamine at a molar ratio of 1:3 to 50, and
the stirring of the 5,5′-bistetrazole-1,1′-diol and the hydroxylamine is performed at a temperature of 40° C. to 60° C.
